(19) United States
(12) Patent Application Publication
(10) Pub. No.: US 2009/0104287 A1
(43) Pub. Date: Apr. 23, 2009

(54) Protocol for AIDS prevention and treatment by nutritional methods

(76) Inventor: Harold Douglas Foster, Victoria (CA)

Correspondence Address: ROBERT H. BARRIGAR;BARRIGAR INTELLECTUAL PROPERTY LAW, 1007 FORT STREET, SUITE 201, VICTORIA, BC, V8V 3K5, CA

(21) Appl. No.: 10/600,028

(22) Filed: Jun. 23, 2003

Related U.S. Application Data
(60) Provisional application No. 60/390,509, filed on Jun. 24, 2002.

(51) Int. Cl. A61K 33/04; A61P 31/18

(52) U.S. Cl. 424/702

(57) ABSTRACT

HIV-1 is a slow virus that has a genome that requires the removal of glutathione, glutathione peroxidase and of selenium, cysteine, glutamine and tryptophan from its host as it is replicated. Although various clinical trials have improved the health of AIDS patients by correcting one or more of these nutritional deficiencies, they have never been addressed together. The current invention combines selenium, cysteine, tryptophan, and glutamine in powder, capsule, or pill form for the creation of glutathione and glutathione peroxidasae by the patient and the correction of deficiencies of other biochemical substances that require selenium, cysteine, tryptophan, and glutamine as precursors. Such a treatment protocol will prevent HIV-1 seropositive individuals from developing AIDS and reverse most of the symptoms of AIDS in those with this disorder.

### FEDERALLY SPONSORED RESEARCH

Not Applicable

### SEQUENCE LISTING OR PROGRAM

Not Applicable

### BACKGROUND

1. Field of Invention

This invention relates to the prevention and treatment of AIDS (acquired immunodeficiency syndrome) through the use of a specific combination of nutrients.

2. Description of Prior Art

As of December 2002 the Joint United Nations Programme on HIV/AIDS (UNAIDS) and the World Health Organization (WHO) estimated that globablly there were 42 million people living with HIV/AIDS. During 2002, 5 million individuals had been newly infected with HIV (human immunodeficiency virus) while 3.1 million died from AIDS (acquired immunodeficiency syndrome). If these trends continue, HIV/AIDS will soon become the greatest catastrophe in human history.

So far, despite enormous scientific effort and expense, no vaccine has been developed that has proven to be effective against HIV-1. The conventional treatment for infection with this virus consists of combinations of nucleoside analog reverse transcriptase inhibitors, nonnucleoside analog reverse transcriptase inhibitors, and the HIV protease inhibitors. These three classes of drugs aim to inhibit the replication of HIV-1, the virus known to cause AIDS. Such drugs do this quite effectively initially, so reducing the death rate from AIDS in regions where they are widely available.

This conventional approach to the treatment of HIV/AIDS has a number of serious disadvantages.

(a) It is very expensive and, as a result, is not widely available to patients in the Developing World where HIV/AIDS is most common.

(b) The drugs involved in conventional treatment are often highly toxic and have been shown to cause a series of adverse side effects, ranging from cardiovascular disease through diabetes to cancer. One of the most widely used drugs, AZT (azidothymidine), for example, is carcinogenic and can be expected to eventually promote cancer in many of the patients treated with it.

(c) HIV-1 is developing resistance to such therapeutic agents since they accelerate viral evolution. One result of this evolutionary process has been the development of dangerous drug-resistant strains of HIV-1 that are now spreading rapidly amongst the highly sexually active and/or drug addicts in North America and Europe. The appearance of such drug resistant strains of HIV-1 in New York, Los Angeles, and Vancouver, for example, constitutes a serious threat to the management of antiretroviral therapy. Indeed, a new strain of HIV-1 that cannot be treated effectively with any existing AIDS drug has begun to spread in Vancouver, British Columbia, Canada. This strain is very resistant to all three classes of inhibitor drugs. Patients infected by it have gone from being totally asymptomatic to having fully developed AIDS in a few months.

(d) As a consequence of the immune system decline seen in HIV/AIDS patients many require treatment with a variety of antibiotic drugs, in addition to the previously described reverse transcriptase and protease inhibitors. Such extensive use of such antibiotics also has driven old pathogens to evolve drug resistant strains. Bacteria are particularly capable of developing drug resistance for two reasons. They multiply rapidly and can transfer resistant genes. The conventional treatment for HIV/AIDS, therefore, is encouraging the diffusion of drug-resistant pathogens such as those that are responsible for typhoid, tuberculosis, and Russia. At the same time, rapid transportation ensures that newly evolving resistant microbial strains are quickly spread. Resistant strains of gonorrhea that developed in Africa and Asia are now found globally.

### OBJECTS AND ADVANTAGES

The current invention involves a treatment protocol that uses only essential nutrients. It includes no antiviral nor antibiotic drugs and is cheap, non-toxic, and will not stimulate the evolution of HIV-1, HIV-2, or other AIDS-associated bacterial or viral pathogens. Accordingly, besides the objects and advantages of the AIDS treatment protocol described in my above patent, several objects and advantages of the present invention are:

(a) To provide a nutritional treatment for HIV/AIDS that is easy to administer and relatively inexpensive so that it can be made far more widely available than current conventional treatments.

(b) To provide a non-toxic alternative to the current conventional drug treatment for HIV/AIDS that, if taken as directed, will not be associated with adverse side effects.

(c) To provide a treatment for HIV/AIDS that will not cause any increase in drug-resistant viral strains.

(d) To provide a treatment for HIV/AIDS that will not cause any increase in drug-resistant bacterial strains.

(e) To provide a treatment that will reverse the symptoms of HIV infection, known as AIDS, and continue such reversal indefinitely.

(f) To provide a treatment for HIV/AIDS that will permit those infected by HIV-1 and/or HIV-2 to live a life of normal length and physical activity, never developing AIDS.

### SUMMARY

In accordance with the present invention the optimum treatment for HIV/AIDS is shown to be replacement of the four nutrients: selenium, cysteine, tryptophan, and glutamine removed from the human body by the replication of HIV-1.

### DRAWINGS

Not Applicable

### DETAILED DESCRIPTION

### Field of Invention

The present invention relates to a composition and method for the nutritional prevention and reversal of AIDS. More particularly, this invention relates to a protocol consisting of powder, capsules, or pills containing the four nutrients—selenium, cysteine, tryptophan, and glutamine—for administration to HIV-AIDS patients by mouth.

### BACKGROUND OF INVENTION

HIV-1 encodes for the human selenoenzyme glutathione peroxidase. As it replicates in the-human body, its genetic needs require it to deprive HIV-1 seropositive individuals not only of glutathione peroxidase, but also of the four basic components of this selenoenzyme, namely selenium, cysteine, tryptophan, and glutamine. Eventually this depletion process causes severe deficiencies of all of these substances, which in turn are responsible for the major symptoms of AIDS. These include immune system collapse, greater susceptibility to cancer and myocardial infarction, muscle wasting, depression, diarrhea, psychosis, and dementia. As deficiencies of these nutrients cause a decline in the efficacy of the immune system, associated pathogenic cofactors, such as the bacteria responsible for tuberculosis, become responsible for their own unique symptoms.

Any treatment for HIV/AIDS, therefore, is going to be far more effective if the protocol involves the normalization of body levels of glutathione, glutathione peroxidase, selenium, cysteine, tryptophan, and glutamine. Although various clinical trials have improved the health and reduced the symptoms of AIDS patients by correcting for one or more of these nutritional deficiencies, such deficiencies have never been addressed or corrected together. The present invention will allow this error to be rectified.

### SUMMARY OF THE INVENTION

The present invention relates to a nutritional powder, capsules, or pills that contain the four nutrients selenium, cysteine, tryptophan, and glutamine.

More particularly, this invention relates to a nutritional composition that contains from about 100 to about 1,000 micrograms of selenium, from about 1 to about 4 grams of cysteine either as N-acetyl-cysteine or selenocyosteine, from about 1 to about 4 grams of tryptophan, and from about 10 to about 25 grams of glutamine. Such a nutritional invention can optionally contain additional vitamins and minerals including vitamins B1, B6, C, and E and minerals magnesium and zinc that act as cofactors in the body's utilization of these four nutrients.

### DETAILED DESCRIPTION OF THE INVENTION

It is currently the conventional wisdom that the “HIV [human immunodeficiency virus] is the sole cause of AIDS.” The processes involved, however, are still poorly understood and there are at least eight current hypotheses that attempt to account for the causal link between HIV-1 and AIDS. Any successful explanation of how HIV-1 infection ultimately leads to AIDS must be able to answer at least four significant questions. Firstly, why are the initial symptoms of HIV-1 infection so minor? Why does AIDS develop long after the production of antibodies to HIV-1, often taking several years for HIV-1 positive individuals to develop the disease? Why are many organs so adversely affected, even when they may show little evidence of any direct negative HIV-1 impact? Fourthly, how can the extremely diverse symptoms of AIDS be caused by one virus? Successful answers to these questions necessitate an understanding of what HIV-1 requires from its host and how such losses affect those who are HIV-1 seropositive? This has been answered, in part, by Taylor and coworkers at the University of Georgia who have demonstrated that HIV-1 encodes for a homologue of one of the human glutathione peroxidases. As a consequence, replication of the virus deprives seropositive individuals not only of this form of the selenoenzyme but also of its four basic components, namely selenium, cysteine, glutamine, and tryptophan.

It is hypothesised here that this depletion process eventually causes severe deficiencies of all of these substances in HIV-1 seropositive individuals. These nutrient inadequacies are, in turn, responsible for most of the symptoms of AIDS. Selenium deficiency, for example, has been linked to the depressed production of CD4 T lymphocytes and to greater susceptibility to cancer and myocardial infarction. Cysteine depression encourages both psoriasis and abnormal immune function, while glutamine deficiency results in depression, diarrhea, and muscle wasting. Inadequate tryptophan in turn causes both dementia and dermatitis (Table 1).

TABLE 1
AIDS: The Viral Induced Nutrient Deficiency Disease

| Nutrient | Deficiency Symptoms (Examples) |
|---|---|
| | AIDS |
| Glutathione | Increased free radical damage, higher |
| | incidence of cancer, heart disease, |
| | premature aging. |
| Glutatione peroxidase | Elevated hydrogen peroxide, oxidative |
| | stress and lipid peroxidation. |
| Selenium | Depressed glutathione peroxidase. |
| | Oxidative stress. Depressed CD4 T |
| | lymphocytes, depressed triiodothyronine |
| | (hypothyroidism). Cancers of lung, |
| | colon, etc. Myocardial infarction, |
| | Kaposi's sarcoma (with HHV-8), |
| | depression. |
| Cysteine | Depressed glutathione and sulphur. Poor |
| | wound and skin healing. Psoriasis. |
| | Abnormal immune function, secondary |
| | infections and cancers. |
| Glutamine | Depression. Abnormal intestine |
| | permeability, diarrhea, muscle wasting. |
| Tryptophan | Depressed niacin and serotonin levels. |
| | Immune incompetence. Neuroendrocrine |
| | disregulation, polyneuropathy, |
| | dementia, dermatitis, diarrhea. |
| | AIDS |

### Nutrient Deficiencies HIV-AIDS

If this viral-induced nutrient deficiency hypothesis is correct then AIDS patients will display inadequacies not just of glutathione and glutathione peroxidase but also of its four components, namely selenium, cysteine, glutamine and tryptophan. Evidence to confirm that this is the case is now presented.

### Glutathione Deficiency

Glutathione deficiency is a common characteristic of HIV-infected individuals and is associated with both impaired T cell function and an accelerated death rate. Interestingly, N-acetyl cysteine supplementation often seems effective in replenishing both whole blood and T cell glutathione in such HIV seropositive patients.

### Glutathione Peroxidase Deficiency

The effects of glutathione peroxidase on inhibiting HIV activation have been well documented. Furthermore, it is apparent that this selenoenzyme becomes depleted in infected individuals. Gil and coworkers, for example, compared the glutathione peroxidase levels in blood taken from 85 HIV/AIDS patients and 40 healthy controls, confirming a significant (P<0.05) reduction of glutathione peroxidase in the infected group.

### Selenium Deficiency

Several studies have shown that there are declining levels of plasma selenium in individuals with HIV/AIDS. Baum and coworkers, for example, monitored 125 HIV-1 seropositive male and female drug users in Miami Florida, establishing that depressed selenium levels were a better. predictor of mortality than CD4 T cell counts. Similarly, 24 HIV-infected children were monitored over 5 years, during which time half of them died of HIV-related causes. Once again, the lower their serum selenium levels, the more rapidly death occurred, indicating an association between selenium deficiency and more rapid disease progression. It is not surprising, therefore, that AIDS has diffused fastest in low selenium regions.

### Cysteine Deficiency

HIV-infected patients also display decreased plasma cysteine concentration at all disease stages. Such deficiencies also have been identified in SIV-infected rhesus macaques. Beyond this, clinical studies have demonstrated that cysteine supplementation (usually given as N-acetyl cysteine) replenishes low glutathione in CD4 T cells, apparently improving the ability to resist secondary pathogens and cancers. Since cysteine is a major source of sulphur, it is not surprising that HIV-infected patients are also very sulphur deficient.

### Glutamine Deficiency

Glutamine is a major requirement of rapidly proliferating cells. As a consequence, it is of great significance in the digestive tract where it is essential for intestinal cell proliferation, intestinal fluid/electrolyte absorption and mitogenic response to growth factors. A lack of glutamine also produces apoptosis. Glutamine deficiency is definitely a characteristic of AIDS and it is not surprising, therefore, that patients suffer from abnormal intestinal permeability and associated digestive malfunction. These symptoms can be corrected by glutamine supplementation.

### Tryptophan Deficiency

It has been demonstrated that, as would be expected if the glutathione peroxidase depletion hypothesis described here is correct, tryptophan is depressed in the serum and cerebrospinal fluids of patients with HIV-infection. Indeed, Werner and coworkers have shown that, in patients with advanced HIV-infection, tryptophan levels are less than 50% of those in gender and age matched controls. Since tryptophan is an essential amino acid that is needed for the biosynthesis of niacin and serotonin, it is not surprising that their levels are also depressed in HIV/AIDS patients.

It is apparent from the references cited above that, just as predicted by the viral-induced nutrient deficiency hypothesis, HIV/AIDS patients are indeed deficient in glutathione and in glutathione peroxidase and its four components selenium, cysteine, glutamine and tryptophan. Furthermore, all these deficiencies worsen as HIV/AIDS progresses.

The current invention will reverse these deficiencies and, with them, many of the symptoms commonly seen in HIV/AIDS patients. Since just how deficient such patients will be in each nutrient will vary, depending on both previous diet and disease progression, the invention will provide such nutrients in powder, capsule, or pill form at non-toxic levels, so that daily dosages can be varied depending on degree of nutrient deficiency. Depending on the severity of the symptoms of HIV/AIDS, it is anticipated that the daily dose of the invention will contain from about 100 to about 1,000 micrograms of selenium, from about 1 to about 4 grams of N-acetyl-cysteine or selenocyosteine, from about 1 to about 4 grams of tryptophan, and from about 10 to about 25 grams of glutamine.

### CONCLUSION, RAMIFICATIONS, AND SCOPE

Since HIV-1 and indeed all viruses that encode for glutathione peroxidase are using selenium, cysteine, glutamine, and tryptophan for their own purposes, those infected by such viruses become extremely deficient in these nutrients. These deficiency states have serious health implications. In humans, selenium, for example, is not only required to produce glutathione peroxidase but is also utilized in 15 other selenoproteins. To illustrate, selenium is an essential component of deiodinase, an enzyme needed to convert thyroxine to triiodothyronine. AIDS patients are deficient in selenium and therefore deiodinase and therefore triiodthyronine. As a result, they have many symptoms caused by thyroid malfunction. Tryptophan is involved in much more than just glutathione peroxidase production. It is also required to produce niacin and serotonin. Since HIV-1 is competing for it, the patient has low tryptophan stores, resulting in depressed niacin and serotonin. The end results of low niacin and serotonin levels include several AIDS symptoms such as dermatitis, diarrhea, and dementia. The same symptoms occur in people who are not HIV-1 positive but simply eat a diet that is very low in tryptophan, causing a disease known as pellagra. In summary, HIV-1 robs the body of selenium, cysteine, tryptophan, and glutamine. As a result, all the various uses of these four nutrients in the human body are poorly met, and an enormous number of deficiency symptoms develop conventionally called AIDS (Table 1).

The current invention, by supplying high levels of the four nutrients selenium, cysteine, glutamine, and tryptophan, allows them to be used either to produce glutathione and glutathione peroxidase or as precursors for other essential biochemical substances. If taken on a daily basis early after infection by HIV-1, the invention will prevent the development of AIDS. If treatment begins after the symptoms of AIDS have developed, the invention will reverse all of those symptoms that are directly or indirectly related to deficiencies of these nutrients.

I claim:

1. A series of nutritional compositions for the treatment of infection by human immunodeficiency viruses that contains the four nutrients required by humans to produce one of the glutathione peroxidases wherein selenium is present in an amount from about 0.0004 to about 0.0008 weight percent, cysteine is present in an amount from about 3.4 to about 37.5 weight percent, tryptophan is present in an amount from about 3.4 to 37.5 weight percent, and glutamine is present in an amount from about 55.0 to about 86.0 weight percent.

2. A method of preventing the development of human immunodeficiency virus infection into acquired immunodeficiency syndrome by administering daily a therapeutically effective amount of the nutritional composition according to claim 1.

3. A method of reversing the symptoms of human immunodeficiency virus infection known as acquired immunodeficiency syndrome by administering daily a therapeutically effective amount of the nutritional composition according to claim 1.
